(19) United States
(12) Patent Application Publication
(10) Pub. No.: US 2007/0178124 A1
(43) Pub. Date: Aug. 2, 2007

(54) Inorganic phosphates reversibly grafted with bioactive compounds and a method of their preparation

(75) Inventors: Vasily S. Spiridonov, Moscow (RU); Pavel G. Mingalev, Moscow (RU); Georgy V. Lisichkin, Moscow (RU); Michael G. Goldfeld, Irvine, CA (US); Anna V. Skliarenko, Moscow (RU); Valentina B. Kurochkina, Moscow (RU); Jenny E. Sattarova, Moscow (RU)

Correspondence Address: Dr. Michael Goldfeld, 63 Summerstone, Irvine, CA, 92614, US

(73) Assignees: Dr. Michael Goldfeld; Advanced Science Application Partnership (A.S.A.P.)

(21) Appl. No.: 11/649,611

(22) Filed: Jan. 4, 2007

(30) Foreign Application Priority Data
Jan. 30, 2006 (RU) 2006102533

(51) Int. Cl. A61K 9/00

(52) U.S. Cl. 424/400

(57) ABSTRACT

Biologically active solid systems are disclosed, containing immobilized organic compounds, such as medicines, in the form of materials appropriate as bone fillers, implant coatings or other alike systems, of the common formula B—P-A, where B is an inorganic support consisting of calcium phosphate, calcium hydroxyphosphate (hydroxyapatite), their mixture, or some other biocompatible mixture containing these or other insoluble inorganic phosphates, —P— is a phosphoryl group —P—O—, and A is the residue of the immobilized, organic, biologically active compound, such as a medicine, containing unprotonated amine and/or hydroxide group(s), and a method of their preparation consisting of the treatment of inorganic phosphate with a grafting reagent and consequent reaction with organic compound. As a grafting reagent, phosphorus pentachloride is applied and all operations are completed in an aprotic solvent. The advantage of the above product is the extension of the time duration of release of the immobilized organic compound such as a medicine from the surface, the release of the organic compound in its initial, unaltered, fully biologically active form, in the absence of any toxic or alien side products of hydrolytic decomposition of the immobilized system.

### FIELD OF THE INVENTION

The invention concerns surface chemistry, in particular chemistry of biocompatible calcium phosphate and other water-insoluble inorganic phosphates or materials containing those, applied in coatings of orthopedic implants, fillers of polymer bone cements, carriers of cell cultures and medicines, materials for environment protection, cleaning and disinfection of aquatic systems and other comparable applications. The invention allows modifying the absorption characteristics of the surface of biocompatible inorganic phosphates, such as, but not limited to, hydroxyapatite (HAP), such modification being then applied to develop medicines of local, targeted, sustained action, by a reversible attachment of medicines, or developing other materials steadily releasing unaltered biologically active organic compounds in the surrounding aqueous systems.

### BACKGROUND OF THE INVENTION

Inorganic phosphates, such as calcium phosphate, are notoriously known as chemically saturated, non-reactive materials, which are hard to modify without total destruction of their crystalline lattice using such harsh conditions as concentrated acids or high-temperature reduction, therefore it is a challenging problem to graft their surface with organic compounds while keeping the core of these organic compounds intact.

In the literature (U.S. Pat. Nos. 6,821,528 and 6,767,550, papers by Pham H. H, Luo P, Genin F, Dash A. K. Synthesis and Characterization of Hydroxyapatite-Ciprofloxacin Delivery Systems by Precipitation and Spray Drying Technique. AAPS PharmSciTech. 2002; 3(1), Murugan R., Rao K. P. Controlled release of antibiotic from surface modified coralline hydroxyapatite. Trends Biomaterials Artif. Organs, v.16 (2002) 4345, A. C. Queiroz, J. D. Santos, F. J. Monteiro, J. C. Knowles, I. R. Gibson Adsorption and release studies of sodium ampicillin from hydroxyapatite and glass reinforced hydroxyapatite. Biomaterials 22 (2001) 1393-1400), several methods have been described for placing various medicines on hydroxyapatite (HAP) implants.

The disadvantage of those methods is rather rapid washing out of the medicines by interstitial body fluid from the surface of implants (from several hours to 1 week, whereas the resorption of implant takes up to 1 year). Application of biopolymers does not substantially improve the situation, besides it is undesirable to introduce alien proteins and non-resorbable materials in the organism.

There are examples of successful modification of HAP surface with various organic compounds (Tanaka H., Watanabe T., Chikazawa M., Kandori K., Ishikawa T. Surface structure and properties of calcium hydroxyapatite modified by hexamethyldisilazane, Journal of Colloid and Interface Science. 1998; 206:205-11; L. Borum, O. C. Wilson, Surface modification of hydroxyapatite. Part II. Silica. Biomaterials 2003; 24:3681-8; Tanaka H., Yasukawa A., Kandori K., Ishikawa T., Surface modifications of calcium hydroxyapatite with hexyl and decyl phosphate. Colloids & Surfaces, 1997; 125:53-62). It was demonstrated that modification of HAP surface can change adsorption characteristics of the material and can be completed under conditions which do not disturb the crystalline structure of calcium phosphate.

A solution closest in its technological substance to the present invention has been described in an article by Ginalzka G., Osin'ska M., Uryniak A., Covalent Method of Gentamicin Bonding to Silica Supports. J. Biomaterials application Nos. 2004, Vol. 18, pp. 279-289. These authors suggest to covalently attaching the Gentamicin antibiotic to a surface which has been previously silanized. Aminopropyltriethoxysilane was used as a grafting agent in this study. The disadvantage of this approach is the necessity to coat the implant surface with an alien silicon-containing grafting agent. Besides, in the course of grafting, the molecular structure of the grafted antibiotic is essentially and irreversibly distorted. Finally, the antibiotic is released from the surface together with the residue of the grafting agent.

### Problems Addressed and Solved by the Invention

The major problems addressed and solved by our invention are:

to develop a method of modification of the surface of various calcium phosphates using compounds which do not release, at their decomposition in the organism, any toxic and/or alien products;

to develop a method of coating such a modified calcium phosphate surface by a medicine or some other biologically active compounds;

to develop a method of such a grafting of organic compound that would provide its reversible bonding, so that it will be released in its unaltered, fully biologically active form;

to develop a method of such a grafting that would provide for an extended time period of organic compound gradual release due to its washing out in an aquatic system, such as interstitial body liquid, so that such a product will have a prolonged time of action.

The above problems are solved in this invention, in particular, in application to immobilized organic compounds such as medicines, in a system whose composition may be presented with a general formula

B—P-A

where B is the basic inorganic phosphate structure such as calcium phosphate or calcium hydroxyphosphate (hydroxyapatite, HAP), their mixture, or any other mixture containing such a phosphate or hydroxyphosphate in its solid phase; P—is a phosphoryl atomic group —P—O— grafted to the surface of the above solid; and A is an organic compound, such as a medicine, residue containing unprotonated amine and/or hydroxy groups, preferably in the amount of 0.5 to 2 mass %.

The above problems are further solved by the method developed of producing immobilized organic compounds, such as medicines, in a system of a general formula B—P-A, which includes the treatment of inorganic phosphate, hydroxyphosphate, their mixtures or other solids containing them, by a grafting reagent, with subsequent interaction with a medicine, such as an antibiotic containing unprotonated amine of hydroxy groups, which is distinct by the application, as a grafting reagent, of phosphorus pentachloride, PCl₅, and all operations are completed in a dry aprotic solvent. It is preferable to apply phosphorus pentachloride in the amount of up to 7 mass % from the amount of calcium or other inorganic phosphate or hydroxyphosphate or their mixtures, or other solids containing them.

To solve the above problems, it is suggested to create an intermediate grafted layer (or film, or coating), on the inorganic substrate, such as calcium phosphate or hydroxyphosphate surface, this grafted film being able to react with organic compounds, such as certain medicines or other biologically active reagents. The effect of extending the duration of the release of the medicine from the surface of the solid is achieved due to the formation of covalent chemical bonds surface—medicine (mostly covalent P—N and/or P—O bonds) which in the presence of water are gradually hydrolyzed (with the formation of P—O—H groups on the surface and recovery of the initial N—H and/or O—H groups in the molecule of the organic compound) so that it is being released into the surrounding aquatic system, such as the release of medicine on the body fluid or tissue, in its initial, unaltered, fully biologically active form.

### DETAILED DESCRIPTION OF THE INVENTION

As a solid surface modifier, it is suggested to apply phosphorus pentachloride, PCl₅. As a solid carrier hydroxyapatite or any other biocompatible calcium or another comparable phosphate or their mixture, or another biocompatible solid material containing the above phosphates in the state of powder, granules of any shape, or as a coating on a metallic, or another implant, or device.

It is desirable that the inorganic phosphate possesses a rather large specific surface area of about 10 m²/g or more. If the amount of medicine which is necessary to be introduced together with the implant is small, the specific surface area of calcium phosphate may be lesser. The organic compound such as a medicine must contain in its molecule unprotonated primary amine or hydroxyl groups. Since the modifying agent is easily hydrolizable, it is essential to exclude contact of the above modifying agent, modified inorganic phosphate, and modified phosphate with grafted medicine before its actual application, with water or any other protic solvent.

The modifying agent, phosphorus pentachloride, PCl₅, is dissolved in a thoroughly purified and desiccated aprotic organic solvent. Modification is performed in the milieu of an organic solvent boiling with a reflux with slight agitation for 4 to 10 hours. As a solvent, it is preferable to use low-boiling saturated hydrocarbons, such as hexane, heptane, octane, petrol ether, or carbon tetrachloride. The most suitable amount of modifying agent is 0.01 to 0.03 mmol per 1 m2 of the inorganic phosphate, such as calcium phosphate surface. The most appropriate amount of solvent is 50 to 100 mL per 1 g of calcium phosphate.

After the completion of the modification procedure, the solvent, together with the residual unreacted modifying reagent is removed, and modified calcium Phosphate, or another solid containing it, is thoroughly washed out (at least 5 to 7 times). As a washing solvent, carefully purified and dry saturated hydrocarbons (such as hexane or petrol ether), or carbon tetrachloride, or chloroform, or methylene chloride, or diethyl ether, or tetrahydrofuran, or some other anhydrous aprotic solvent can be applied. It is unacceptable to use, for washing the modified solid, water, alcohols, ketones, etc.) The residual washing fluid is removed by vacuum hot filtration and/or vacuum evaporation. This dried modified calcium phosphate, or material containing it, is placed in a sealed container where it can be stored without deterioration indefinitely. All above operations on preparation and modification of calcium phosphate should be completed in a stream of air, nitrogen, or some other inert gas thoroughly dried out from the traces of humidity in the ambient air.

The active component of the medicine is coated on thus modified calcium phosphate, or a material containing it, using the following procedure.[0016] The medicine is dissolved in a thoroughly purified and dried aprotic solvent, such as dimethylsulfoxide (DMSO), diethyl ether, chloroform, or petrol ether). It is necessary that the active component of the medicine contains unprotonated primary amine and/or hydroxyl groups. It is undesirable to use medicines as their salt of inorganic acids, such as hydrochloride, sulfate, etc. The amount of the medicine should be preferably 0.002 to 0.03 mmol per 1 m2 of the surface area of the modified calcium phosphate. The amount of solvent is determined out of the amount of the organic stuff and calcium phosphate (preferably 10 to 100 mL of solution per 1 g of modified calcium phosphate).

Modified calcium phosphate is placed into the solution of an organic compound possessing amine or hydroxyl group(s), such as a medicine, and kept there for 1 to 10 hours with slight agitation. Then, the solvent with residual unreacted organic stuff is removed and the modified calcium phosphate coated with medicine is washed out several times with appropriate solvents. The solvents used for the said washing are the same solvent that has been applied in the grafting step, and then with diethyl ether, ethyl alcohol or isopropyl alcohol, and water (5 times each solvent). The residual washing solvent is removed by hot vacuum filtration or evaporated under vacuum. Dried modified calcium phosphate-containing solid is packed in a sealed container or applied as needed.

When appropriate reagents are used and all the above requirements to the procedures are fulfilled, the final product is a material with the bulk crystalline structure of the initial calcium phosphate, hydroxyphosphate or a solid containing these, on the surface of which there is a condensed coating, or film, of covalently grafted organic molecules such as a medicine. The product does not contain any residual organic solvents applied at the intermediate steps. The organic compound grafted to the surface of the preliminary modified solid is gradually released, during an extended period of time, due to the slow hydrolysis reaction with water in the interstitial body fluid in its initial protonated form, with no changes in its chemical composition. The same hydrolysis reaction converts the involved inorganic bridging groups at the surface into a bonded phosphate anion remaining at the surface of the implant.

The essentials of the invention are demonstrated below with the following examples in which aminoglycoside antibiotic Gentamicin was used as a representative medicine, and two unprotonated amines were taken as representatives of other organic compounds of appropriate structure.

### EXAMPLE 1

1 g of HAP with its specific surface area of 60 m²/g, was placed in a three-neck round-bottom flask and dried out from the residual humidity under vacuum (0.5 mm Hg, 150° C. for 1 hour), the flask was filled with air dried over roasted calcium chloride. Thus dried HAP was refluxed with 1.3 mmol of PCl₅ dissolved in 85 mL of carbon tetrachloride for 8 hours at slight stirring. After cooling, the solution was decanted; the precipitate was washed out 3 times with dry hexane, carbon tetrachloride and methylene chloride and then dried out under vacuum (0.5 mm Hg) on a water bath. All operations were performed in a stream of air dried over roasted calcium chloride to prevent the hydrolysis of modifying reagent by atmospheric humidity.

X-ray diffraction analysis revealed no peaks of admixture phases, confirming that the three-dimensional structure of the bulk material was not altered. The content of carbon in this intermediate material was under 0.01 mass % (the lower limit of analytical sensitivity). In the same way, the amorphous calcium phosphate (ACP) has been modified. After that step, the product retained its amorphous structure and did not contain any carbon.

### EXAMPLE 2

Modified calcium phosphate supports or carriers prepared in the Example 1 were placed in solutions of octadecylamine (ODA) and tetraethylenepentamine (TEPA) in toluene for 8 hours with slight stirring at room temperature. The above amines were taken in the amount of 0.12 g ODA in 60 mL toluene and 0.2 mL TEPA in 50 mL toluene per 1 g of support. All procedures with chemically of 16modified calcium phosphates were completed under the stream of air dried over roasted calcium chloride. Then, the solutions were decanted, the precipitate was thoroughly washed out with hexane and carbon tetrachloride and the traces of solvents removed under vacuum on water bath. For comparison, the same amines were applied to non-modified calcium phosphate supports. amines were

### EXAMPLE 3

50 mg of amorphous calcium phosphate and 50 mg of HAP, coated with ODA and TEPA from the Example 2, were placed in 50 mL of distilled water and kept there with periodical stirring at room temperature for various time periods. The precipitate was filtered and dried under vacuum on a water bath. The content of ODA and TEPA on the support was determined upon the content of carbon using standard procedures of elemental analysis. The data thus obtained, reflecting the gradual release of the above amines from the support as a result of hydrolysis, are summarized in Table 1 to 4. The amount of ODA and TEPA is indicated as the number of molecules of these compounds per 1 nm2 of the surface area.

TABLE 1
Hydrolytic release of ODA from HAP surface.

| | Desorption time (min) | | | |
|---|---|---|---|---|
| | 0 | | | |
| | (before hydrolysis) | 1 | 4 | 15 |
| HAP/PCl₅ | 4.2 ± 0.05 | 4.2 ± 0.05 | 4.1 ± 0.05 | 3.6 ± 0.05 |
| Unaltered HAP | 5.0 ± 0.05 | 4.5 ± 0.05 | 3.4 ± 0.05 | 2.7 ± 0.05 |

TABLE 2
Hydrolytic release of TEPA from HAP surface.

| | Desorption time (min) | | | |
|---|---|---|---|---|
| | 0 | | | |
| | (before hydrolysis) | 1 | 4 | 15 |
| HAP/PCl₅ | 4.2 ± 0.05 | 4.2 ± 0.05 | 4.1 ± 0.05 | 3.6 ± 0.05 |
| Unaltered HAP | 5.0 ± 0.05 | 4.5 ± 0.05 | 3.4 ± 0.05 | 2.7 ± 0.05 |

TABLE 3
Hydrolytic release of ODA from ACP surface.

| | Desorption time (min) | | | |
|---|---|---|---|---|
| | 0 | | | |
| | (before hydrolysis) | 15 | 60 | 240 |
| ACP/PCl₅ | 1.4 ± 0.05 | 1.2 ± 0.05 | 1.0 ± 0.05 | 1.0 ± 0.05 |
| Unaltered AφK | 1.9 ± 0.05 | 1.8 ± 0.05 | 1.4 ± 0.05 | 1.1 ± 0.05 |

TABLE 4
Hydrolytic release of TEPA from ACP surface.

| | Desorption time (min) | | | |
|---|---|---|---|---|
| | 0 (no hydrolysis) | 15 | 60 | 240 |
| ACP/PCl₅ | 3.2 ± 0.1 | 1.3 ± 0.1 | 0.9 ± 0.1 | 1.3 ± 0.1 |
| Unaltered ACP | 1.5 ± 0.1 | 0.9 ± 0.1 | 0.3 ± 0.1 | 0 |

### EXAMPLE 4

1 g of Gentamicin-sulfate (active component content 600 mg/g) was dissolved in 20 mL of distilled water. Sulfate was precipitated with a saturated solution of calcium hydroxide converting Gentamicin into its unprotonated form. The solution was filtered and solvent evaporated under vacuum on a water bath. The antibiotic thus prepared was dissolved in 250 mL of dry dimethylsulfoxide (DMSO). The solution contained 0.23 g Gentamicin per 100 mL DMSO. Modified with phosphorus pentachloride sample of calcium phosphate support from the Example 1, in the amount of 1 g, was placed in 100 mL of Gentamicin solution in DMSO (GENTA/DMSO system) for 4 hours at room temperature and gentle stirring. The solution was decanted, the precipitate washed out consequently with methylene chloride, diethyl ether and distilled water and dried under vacuum on a water bath. For comparison, the same amount of Gentamicin was placed on unaltered samples of calcium phosphate supports.

### EXAMPLE 5

Samples of 100 mg of HAP coated with Gentamicin, from the Example 4, were placed in 10 and 50 mL of distilled water and kept there with gentle stirring at room temperature for various time periods. The precipitate then was filtered and dried under vacuum on a water bath. Gentamicin content on the surface of support was determined upon carbon content. The data are summarized in Table 5 as the mass of Gentamicin, in milligram, per 1 g of HAP.

TABLE 5
Hydrolytic release of GENTA/DMSO from HAP surface.

| | Desorption time (hours) | | | | | | | | |
|---|---|---|---|---|---|---|---|---|---|
| | 0 | | | | | | | | |
| | (no | | | | | | | | |
| | hydrolysis) | 0.02 | 0.25 | 1 | 4 | 16 | 72 | 168 | 288 |
| HAP (HAP/water = 1/100) | 37 ± 1 | — | 28 | 21 | 17 | 11 | 7 | 0 | — |
| HAP (HAP/water = 1/500) | 37 ± 1 | 23 | 17 | 12 | 1 | 3 | — | — | — |
| HAP/PCl₅ (1/100) | 37 ± 1 | — | 26 | 25 | 25 | 27 | | 19 | 19 |
| HAP/PCl₅ (1/500) | 37 ± 1 | 25 | 26 | 26 | 28 | — | 19 | — | — |

### EXAMPLE 6

100 mg HAP with coated Gentamicin from the Example 4 were placed in 10 mL of 0.1 M phosphate buffer solutions, pH 5.5 and &.0, and kept with periodic stirring at room temperature for various time intervals. The precipitate was then filtered and vacuum dried on a water bath. The amount of coated Gentamicin was determined upon carbon content, as before. The data are summarized in Table 6 and 7 as the amount of Gentamicin in milligram per 1 g of HAP.

TABLE 6
Release of Genta/DMSO into phosphate buffer pH 7.0

| | Desorption time (hours) | | | | |
|---|---|---|---|---|---|
| | 0 | | | | |
| | (no | | | | |
| | hydrolysis) | 1 | 4 | 16 | 72 |
| HAP | 27 ± 1 | 26 | 17 | 10 | 2 |
| HAP/PCl₅ | 32 | 27 | 14 | 14 | 13 |

TABLE 7
Release of Genta/DMSO into phosphate buffer pH 5.5

| | Desorption time (hours) | | | | |
|---|---|---|---|---|---|
| | 0 | | | | |
| | (no | | | | |
| | hydrolysis) | 1 | 4 | 16 | 72 |
| HAP | 27 ± 1 | 19 | 15 | 13 | 5 |
| HAP/PCl₅ | 27 | 17 | 17 | 16 | 16 |

### EXAMPLE 7

0.13 g of Gentamicin sulfate (60% of active component) was dissolved in 10 mL of distilled water (GENTA/W). 1 g of modified support from example 1 was placed in this solution and stirred for 4 hours at room temperature, then filtered (this product is denoted as a product of □free adsorption□), or the solvent was evaporated under vacuum on a water bath (□forced adsorption□). For comparison, similar operations were completed with unaltered calcium phosphate support.

### EXAMPLE 8

250 mg HAP coated with Gentamicin from the Example 7 were placed in 2.5 mL of distilled water and kept there with periodic stirring at room temperature for various time periods. The amount of coating Gentamicin was determined upon carbon content, as before. The data are summarized in Table 8 as the amount of Gentamicin in milligram per 1 g of HAP.

TABLE 8
Release of Genta/W from HAP surface.

| | Desorption time (min) | | | |
|---|---|---|---|---|
| | 0 (no hydrolysis) | 15 | 60 | 240 |
| HAP (free adsorption) | 12 ± 5 | 0 ± 2 | — | — |
| HAP (forced | 78 ± 7 | 10 ± 5 | 0 ± 2 | — |
| adsorption) | | | | |
| HAP/PCl₅ (free) | 27 ± 5 | 16 ± 5 | 9 ± 5 | 0 ± 2 |
| HAP/PCl₅ (forced) | 78 ± 7 | 43 ± 6 | 11 ± 5 | 4 ± 2 |

What is claimed:

1. Biocompatible and bioactive solid materials of the common formula B—P-A, where B is an inorganic support consisting of calcium phosphate, calcium hydroxyphosphate (hydroxyapatite), their mixture or some other biocompatible mixture containing these or other insoluble inorganic phosphates, —P— is the phosphoryl group —P—O—, and A is the residue of a bioactive organic compound such as a medicine, containing unprotonated amine and/or hydroxide group(s).

2. Immobilized systems, such as medicines, from claim 1 containing 0.5 to 2 mass percent of phosphoryl groups —P—.

3. Method of preparation of immobilized organic systems such as medicines of the common formula B—P-A, from claim 1, including the treatment of calcium phosphate or hydroxyphosphate, or their mixture, or another mixture containing those or other insoluble inorganic phosphate, with a grafting reagent with consequent reaction with biologically active organic compounds containing unprotonated amine and/or hydroxide groups in which the grafting reagent is phosphorus pentachloride, PCl₅, and all operations are performed in an aprotic solvent.

4. Method from claim 3 in which phosphorus pentachloride is used in the amount of up to 7 mass percent out of the amount of calcium phosphate, or another insoluble inorganic phosphate or hydroxyphosphate or their mixture.
